(19) United States
(12) Patent Application Publication
(10) Pub. No.: US 2021/0137990 A1
(43) Pub. Date: May 13, 2021

(54) METHODS FOR EXP ANDING ADIPOSE-DERIVED STEM CELLS

(71) Applicant: CELLECT BIOTHERAPEUTICS LTD., Kfar Saba (IL)

(72) Inventors: Ina SAREL STERN, Ramat Gan (IL); Shai YARKONI, Kfar Saba (IL)

(21) Appl. No.: 17/053,200

(22) PCT Filed: May 7, 2019
(86) PCT No.: PCT/IL2019/050517

§ 371 Date: Nov. 5, 2020

Related U.S. Application Data
(60) Provisional application No. 62/668,341, filed on May 8, 2018.

(51) Int. Cl. A61K 35/28; C12N 5/0775; A61K 35/32

(52) U.S. Cl. CPC A61K 35/28; C12N 5/0667; C12N 2506/1346; C12N 2501/25; C12N 2501/48; A61K 35/32

(57) ABSTRACT

The invention concerns methods for propagating mesenchymal stem cells (MSC), and in particular adipose derived stem cells, comprising incubating cells isolated from a body tissue with at least one TNF superfamily ligand, and at least one apoptosis inhibitory agent. The cells can be used for transplantation into subjects in need thereof or be induced to differentiate into various cell types that can be used in transplantation.

### TECHNOLOGICAL FIELD

The invention is in the field of cell transplantation, and in particular the invention provides methods for propagating adipose-derived stem cells in vitro.

### BACKGROUND ART

References considered to be relevant as background to the presently disclosed subject matter are listed below:
(1) Le Gallo M, Poissonnier A, Blanco P, Legembre P. Cd95/fas, non-apoptotic signaling pathways, and kinases. Frontiers in Immunology 2017; 8:1216.
(2) Rippo M, Babini L, Prattichizzo F, Graciotti L, Fulgenzi G, Ardori F T, et al. Low fasl levels promote proliferation of human bone marrow-derived mesenchymal stem cells, higher levels inhibit their differentiation into adipocytes. Cell death & disease 2013; 4:e594.
(3) Ham O, Lee S-Y, Song B-W, Cha M-J, Lee C Y, Park J-H, et al. Modulation of fas-fas ligand interaction rehabilitates hypoxia-induced apoptosis of mesenchymal stem cells in ischemic myocardium niche. Cell transplantation 2015; 24:1329-41.
(4) Fan V H, Au A, Tamama K, Littrell R, Richardson L B, Wright J W, et al. Tethered epidermal growth factor provides a survival advantage to mesenchymal stem cells. Stem cells 2007; 25:1241-51.
(5) Rodrigues M, Blair H, Stockdale L, Griffith L, Wells A. Surface tethered epidermal growth factor protects proliferating and differentiating multipotential stromal cells from fasl-induced apoptosis. Stem Cells 2013; 31:104-16.
(6) Rodrigues M, Turner O, Stolz D, Griffith L G, Wells A. Production of reactive oxygen species by multipotent stromal cells/mesenchymal stem cells upon exposure to fas ligand. Cell transplantation 2012; 21:2171-87.
(7) Kennea N, Stratou C, Naparus A, Fisk N, Mehmet H. Functional intrinsic and extrinsic apoptotic pathways in human fetal mesenchymal stem cells. Cell Death & Differentiation 2005; 12:1439-41.
(8) Mizuno H, Tobita M, Uysal A C. Concise review: Adipose-derived stem cells as a novel tool for future regenerative medicine. Stem cells 2012; 30:804-10.
(9) Ravid O, Shoshani O, Sela M, Weinstock A, Sadan T W, Gur E, et al. Relative genomic stability of adipose tissue derived mesenchymal stem cells: Analysis of ploidy, h19 long non-coding ma and p53 activity. Stem cell research & therapy 2014; 5:1.
(10) Sela M, Tirza G, Ravid O, Volovitz I, Solodeev I, Friedman O, et al. Nox1-induced accumulation of reactive oxygen species in abdominal fat-derived mesenchymal stromal cells impinges on long-term proliferation. Cell death & disease 2015; 6:e1728.
(11) Shoshani O, Zipori D, Shani N. The tissue specific nature of mesenchymal stem/stromal cells: Gaining better understanding for improved clinical outcomes. RNA & DISEASE 2015; 2.
(12) Guo J, Nguyen A, Banyard D A, Fadavi D, Toranto J D, Wirth G A, et al. Stromal vascular fraction: A regenerative reality? Part 2: Mechanisms of regenerative action. Journal of Plastic, Reconstructive & Aesthetic Surgery 2015.
(13) Nguyen A, Guo J, Banyard D A, Fadavi D, Toranto J D, Wirth G A, et al. Stromal vascular fraction: A regenerative reality? Part 1: Current concepts and review of the literature. Journal of Plastic, Reconstructive & Aesthetic Surgery 2015.
(14) WO 2007/138597
(15) WO 2017/051421
(16) Barnhart B C, Legembre P, Pietras E, Bubici C, Franzoso G, Peter M E. Cd95 ligand induces motility and invasiveness of apoptosis-resistant tumor cells. The EMBO journal 2004; 23:3175-85.
(17) Desbarats J, Birge R B, Mimouni-Rongy M, Weinstein D E, Palerme J-S, Newell M K. Fas engagement induces neurite growth through erk activation and p35 upregulation. Nature Cell Biology 2003; 5.
(18) Letellier E, Kumar S, Sancho-Martinez I, Krauth S, Funke-Kaiser A, Laudenklos S, et al. Cd95-ligand on peripheral myeloid cells activates syk kinase to trigger their recruitment to the inflammatory site. Immunity 2010; 32:240-52.
(19) Matsumoto N, Imamura R, Suda T. Caspase-8- and jnk-dependent ap-1 activation is required for fas ligand-induced it-8 production. The FEBS journal 2007; 274:2376-84.
(20) Tauzin S, Chaigne-Delalande B, Selva E, Khadra N, Daburon S. The naturally processed cd951 elicits a c-yes. Calcium/PI3K-Driven Cell 2011.

Acknowledgement of the above references herein is not to be inferred as meaning that these are in any way relevant to the patentability of the presently disclosed subject matter.

### BACKGROUND

Fas ligand (Fas-L), a member of the tumor necrosis factor (TNF) family, is a type II transmembrane protein expressed on the surface of immune cells, such as lymphocytes, natural killer (NK) cells and macrophages. Upon binding to its receptor (CD95 or Fas receptor (FasR)), Fas-L induces apoptosis. Fas-L has long been considered a death ligand that promotes apoptosis by binding to its receptor and activating caspases. However, accumulating data now suggest that Fas can transmit various non-apoptotic signals that may have far-reaching implications on a range of physiological and disease conditions [1]. Fas-L has been implicated in immune system homeostasis maintenance and has been suggested to be a guardian against autoimmunity. Yet, increasing evidence demonstrate involvement of Fas-L signaling in onset of additional cellular responses, such as inflammation, proliferation, regeneration, and cancer progression. The relatively ubiquitous expression of CD95 (FasR) in a variety of cells and tissues, corroborates this new line of thought.

Mesenchymal stromal cells (MSCs) are multipotent cells that can be produced from most adult body tissues. MSCs are characterized by their adherence to plastic, their ability to differentiate, in culture, to bone, fat and cartilage, and their expression of a set of distinct surface markers. Use of MSCs for various regenerative and immunosuppressive clinical indications has been suggested and their efficacy is currently being evaluated in numerous clinical trials. Despite their considerable potential, transition of MSC use into routine clinical practice has not been achieved to date, partly due to their poor long-term survival following administration.

Low Fas-ligand (FasL) levels promote proliferation of MSCs derived from human bone marrow and higher levels inhibit their differentiation into adipocytes [2]. As MSCs are known to migrate to inflamed and damaged regions and since they are known to express CD95, it was previously suggested that Fas-L-induced apoptosis may play a major role in their rapid death in vivo [3]. This notion is further supported by the high Fas-L-dependent apoptosis rates observed in cultured bone marrow (BM) [4-6] and fetal blood [7] MSCs.

Adipose-derived stem cells (ASCs), are MSCs derived from the stromal vascular fraction (SVF) of subcutaneous fat. While they have been shown to share all characteristics and most of the regenerative and immunosuppressive properties described for bone marrow (BM) MSCs [8], they also display tissue-specific characteristics [9-11]. Freshly isolated SVF has recently been suggested as an alternative to cultured ASCs to be used within the surgical arena, immediately following their harvest, for regenerative and immunosuppressive purposes. Importantly, the use of freshly isolated autologous SVF for cellular therapies will significantly decrease the cost and time of treatment and regulatory burden compared to cultured ASCs [12, 13]. The therapeutic efficacy of SVF transplantation has been established in various pre-clinical disease models and its use for various clinical indications is currently being evaluated in clinical trials [13].

WO 2007/138597 [14] relates to a method of selecting stem cells from a heterogeneous population of cells isolated from bone marrow by contacting the population of cells with a pro apoptotic agent.

WO 2017/051421 [15] discloses methods for propagating mesenchymal stem cells (MSC), and particularly adipose derived stem cells, comprising incubating isolated cells obtained from a tissue or organ comprising MSC in a growth medium comprising an apoptosis inducing agent.

### GENERAL DESCRIPTION

In a first of its aspects, the present invention provides a method for propagating mesenchymal stem cells (MSC), the method comprising:
(a) isolating cells from a body tissue or organ; and
(b) incubating the isolated cells obtained in (a) in a growth medium comprising:
   (i) at least one TNF superfamily ligand, and
   (ii) at least one apoptosis inhibitory agent,

thereby obtaining a cell population enriched with MSC.

In another aspect, the present invention provides a method for expanding adipose derived stem cells (ASC), the method comprising:
(a) isolating stromal vascular fraction (SVF) cells from a liposuction aspirate; and
(b) incubating the isolated cells obtained in (a) in a growth medium comprising:
   (i) at least one TNF superfamily ligand, and
   (ii) at least one apoptosis inhibitory agent,

thereby obtaining a cell population enriched with ASC.

In one embodiment, prior to step (b) said SVF cells are maintained in culture for at least two passages.

In some embodiments, said TNF superfamily ligand is selected from the group consisting of Fas-ligand (FasL), tumor necrosis factor (TNF) a, TNF-related apoptosis-inducing ligand (TRAIL), tumor necrosis factor-like weak inducer of apoptosis (TWEAK), and a combination thereof.

In one specific embodiment, said TNF superfamily ligand is FasL.

In some embodiments, said growth medium further comprises an additional active agent selected from the group consisting of a growth factor, a hormone, a cytokine and any combination thereof.

In one embodiment, said incubation is for at least 1 hour.

In some embodiments, said incubation is for about 1 hour, 2 hours, 3 hours or more, or for about 1, 2, 3, 4, 5, 6, 7, 8, 9, 10, 11, 12, 13, 14, 17, 20, or 23 days.

In one embodiment, said incubation is for 2 hours.

In one embodiment, said incubation is for 4 days.

In one embodiment, said incubation is for 14 days.

In one embodiment, the medium is refreshed every 3 days or every 4 days. In one embodiment, said apoptosis inhibitory agent is administered prior to, together with, or after said at least one TNF superfamily ligand.

In some embodiments, said apoptosis inhibitory agent is selected from the group consisting of a caspase inhibitor, a serine protease inhibitor (e.g. HTRA2), inhibitors of apoptosis proteins (IAP) (e.g. XAF1), NF-kappa B inhibitor, a Smac inhibitor, and a combination thereof.

In one specific embodiment, said caspase inhibitor is Z-VAD-fmk.

In some embodiments, the amount of said apoptosis inhibitory agent is in the range of about 1 μM to about 50 μM, or in the range of about 5 μM to about 25 μM.

In some embodiments, the concentration of said at least one TNF superfamily ligand is in the range of about 0.1 ng/ml to about 100 ng/ml.

In one embodiment, following incubation with said at least one TNF superfamily ligand and the apoptosis inhibitory agent, said at least one TNF superfamily ligand and the apoptosis inhibitory agent are removed and said cells are allowed to differentiate.

In one embodiment, said cells are allowed to differentiate into adipocytes, chondrocytes, or osteocytes.

In one embodiment, the method of the invention further comprises transplanting the differentiated or undifferentiated cells into a patient in need thereof.

In one embodiment, said cells differentiate into fat tissue, bone tissue, muscle, connective tissue or cartilage.

In one embodiment, said cells differentiate into fat tissue and said patient is in need of breast restoration.

In one embodiment, said cells differentiate into bone tissue and said patient is in need of treatment of orthopedic injuries, bone reconstruction or bone repair.

In one embodiment, said cells differentiate into cartilage tissue and said patient is in need of chondrocyte implantation, treatment of meniscus, rotator cuff or an articular cartilage repair.

In one embodiment, said patient is in need of a cosmetic procedure.

In one embodiment, said transplanting said differentiated cells is for treating a dermatological condition.

In one embodiment, said transplanting said differentiated cells is for alleviating or treating an immune-related disease in a patient.

In one embodiment, said patient undergoes organ or tissue transplantation.

In one embodiment, said organ transplantation is selected form the group consisting of bone marrow, liver, kidney, blood vessel or heart transplantation.

In one embodiment, said transplanting said differentiated cells is for treating cardiac disorders in a patient.

In one embodiment, said cardiac disorders are selected from the group consisting of stroke, angina and heart failure.

In one embodiment, said transplanting said differentiated cells is for treating anal or perianal fistulas in a Crohn's disease patient.

In one embodiment, said transplanting said differentiated cells is for blood vessel repair.

In some embodiments, said cells are allogeneic or autologous.

In another aspect, the present invention provides an adipose stem cell (ASC) growth medium or a mesenchymal stem cell (MSC) growth medium comprising a cell culture medium, a TNF superfamily ligand and an apoptosis inhibitory agent.

In one embodiment, the ASC or MSC growth medium further comprises serum or serum substitutes.

In another aspect, the present invention provides an article of manufacture comprising:

a. a vessel containing an adipose stem cell growth medium or mesenchymal stem cell growth medium, wherein said growth medium comprises a cell culture medium, a TNF superfamily ligand and an apoptosis inhibitory agent; and

b. instructions for using the growth medium for expanding adipose stem cells or mesenchymal stem cells in vitro.

In one embodiment, said growth medium further comprises serum or serum substitutes.

The present invention also provides an enriched population of cells obtained by the methods of the invention.

In some embodiments, the enriched population of cells is for use in a transplantation procedure into a patient in need thereof.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1A is a graph showing SVF cell number fold change as a function of Fas-L concentration (ng/ml) as calculated by standard cell counts; FIG. 1B is a graph showing sub-G1 rate fold change as a function of Fas-L concentration (ng/ml) as calculated by standard cell counts; FIG. 1C and FIG. 1D are graphs showing sub-G1 proportions in control SVF cells (without Fas-L treatment) (1C) and in cells treated with 50 ng/ml Fas-L (1D) as calculated by propidium iodide (PI) cell-cycle flow cytometry analysis. Data are presented as the mean±standard deviation. FIGS. 1E and 1F show images of cultured SVF cells either with 50 ng/ml Fas-L (1F) or without Fas-L (control) (1E). FIG. 1G is a graphical representation of annexin/PI flow cytometry analysis of floating cells of treated cells (marked by arrow in FIG. 1F). FIGS. 1H and 1I are graphical representations of control cells (1H) and Fas-L treated cells (FIG. 1I) stained with anti-CD95.

FIG. 2 is a graphic representation of various surface markers expression in human SVF cells at passage (P) 0 (FIGS. 2A-H) and P1 (FIGS. 2I-L), cultured under normal culture conditions (control) (FIGS. 2A-2C, 2G, 2I and 2J) or with 50 ng/ml Fas-L (FIGS. 2D-2F, 2H, 2K and 2L). The cells were examined for their surface marker expression of CD45, CD31, CD34, CD29, CD105 and CD73 by flow cytometry. Staining for CD45 and CD31 is presented in correlation with granularity (side scatter SSC-A)

FIGS. 3A and 3B are photographs of stained cells that differentiated into fat (A—control with no addition of FasL; B—cells incubated with FasL). FIG. 3C is a graph showing the amount of stain shown as optical density (OD). FIGS. 3D and 3E are photographs of stained cells that differentiated into bone (D—control with no addition of FasL; E—cells incubated with FasL). FIG. 3F is a graph showing the amount of stain shown as optical density (OD).

FIG. 4A is a photograph of culture plates with human SVF cultured in the presence or absence (control) of 50 ng/ml Fas-L. Colonies were stained by Giemsa. FIG. 4B is a photograph of an exemplary large colony. FIG. 4C is a photograph of an exemplary small colony. FIG. 4D is a graph showing the total number of colonies. FIG. 4E is a graph showing the number of large colonies (>100 cells) as compared between Fas-L-treated and untreated (control) cells.

FIG. 5A is a graph showing the cell no. fold change of ASCs (P3) that were cultured for 6 days under normal culture conditions or with increasing Fas-L concentrations. FIG. 5B-5D are graphs showing the sub-G1 proportions of the cells. Cell numbers were examined by standard cell counts (5A) and by propidium iodide (PI) cell-cycle flow cytometry analysis (5B-5D). Data (5B) are presented as the mean±standard deviation. FIG. 5E-J are graphs showing surface marker expression of CD29, CD105 and CD73 as examined by flow cytometry in P3 ASCs cultured under normal culture conditions or with 50 ng/ml and 100 ng/ml Fas-L.

FIG. 6A is a graph showing sub-G1 proportions (sub-G1 rate fold change) in ASCs that were treated for 4 days with Fas-L, the caspase inhibitor Z-VAD-fmk or both. FIG. 6B is a graph showing cell number fold change. FIG. 6C-6D also show sub-G1 proportions in control (6C), Fas-L treated cells (6D) and Fas-L+ inhibitor treated cells (6E). The sub-G1 proportions were determined by PI cell-cycle flow cytometry analysis and the cell numbers were determined by standard cell counts. Data (6A-B) are presented as the mean±standard deviation. FIG. 6F-6I are representative images of ASCs under the different conditions.

FIG. 7A-7C are graphs showing the cell no. fold change in cultured ASCs that were treated with the indicated combination of Fas-L, Z-VAD-fmk, LY294002 (PI3K inhibitor) (7A), AZD6244 (ERK-1/2 inhibitor) (7B) and SP600125 (JNK inhibitor) (7C). Data are presented as the mean±standard deviation.

### DETAILED DESCRIPTION OF EMBODIMENTS

The present invention is based on the surprising finding that Fas-L treatment promotes simultaneous apoptosis and proliferation of adipose-derived stem cells (ASC) leading to increased cell yield and phenotypic shift. The understanding of the complex effect of Fas-L on the cells lead the inventors to formulate a novel method for expanding adipose-derived stem cells comprising exposure of the cells to a combination of agents, including Fas-L and an agent that inhibits apoptosis.

As will be elaborated in the Examples below, human ASCs were isolated from routine liposuction procedures and their reactivity to Fas-L treatment was examined ASCs responded to Fas-L by simultaneous apoptosis and proliferation, which yielded a net doubling of cell quantities and a phenotypic shift, including reduced expression of CD105 and increased expression of CD73, in association with increased bone differentiation potential. Treatment of freshly isolated ASCs also led to an increase in large colony forming unit fibroblasts (CFU-F), likely produced by early stem cell progenitor cells.

Interestingly, caspase inhibition by Z-VAD-fmk attenuated Fas-L-induced apoptosis without impacting proliferation, while inhibition of PI3K and MEK, but not of JNK, attenuated Fas-L-dependent proliferation, but not apoptosis. Thus, the inventors show that Fas-L signaling in ASCs leads to their expansion and phenotypic shift through a delicate balance between induced apoptosis and proliferation.

Surprisingly, the inventors found that Fas-L signaling simultaneously activates apoptosis and proliferation of both freshly derived and cultured ASCs. Despite apoptosis induction, Fas-L signaling led to increased cell expansion (˜doubled cell quantities) of ASCs and to a phenotypic shift, evidenced by a change in MSC-specific marker expression, ability to produce higher quantities of early CFU-Fs and increased bone differentiation potential. Further assessments demonstrated that the apoptotic and proliferative Fas-L cues were not interconnected and that blocking the apoptotic signal had no effect on the proliferative signal and vice versa. Proliferation activation was attenuated by both PI3K and MEK inhibitors, indicating the involvement of these pathways in the transmission of the Fas-L signal. In light of the increasing application of both SVF cells and ASCs for regenerative purposes, the use of Fas-L activation combined with inhibition of apoptosis can serve as an efficient tool to increase cell yields and to possibly also augment cell phenotypes for specific clinical indications, such as bone regeneration. Given the drastic apoptotic response and reduced cell yield previously reported following Fas-L treatment of BM MSCs [2, 4-6], the ability of ASCs to respond to Fas-L signaling by a proliferative burst may serve as a clinical advantage upon their delivery, particularly when exposed to Fas-L-presenting immune cells commonly enriched in inflamed areas.

The present invention demonstrates an overall increase in SVF derived and isolated ASC counts upon Fas-L signaling, due to simultaneous apoptosis and proliferation activities.

Fas signaling in ASCs also resulted in a phenotypic change demonstrated by an increase in the percent of cells with low surface expression of CD105 (CD105-low) and high surface expression of CD73 (CD73-high). CD105 and CD73 are both well-established MSC markers and have also been specifically defined as ASC markers. Fas-L treatment led to both an increase in CD105 low ASCs and to their improved bone differentiation potential compared to untreated control.

In order to elucidate the signaling pathways that promote Fas-L-induced apoptosis and/or proliferation, inhibitors of various pathways that were previously demonstrated to promote Fas-controlled apoptotic and nonapoptotic signaling, were utilized. Importantly, the inventors found that inhibition of caspases by the pan-caspase inhibitor Z-VAD-fmk led to near-complete inhibition of apoptosis in Fas-L-treated ASCs but had a minor to negligible effect on proliferation. This strongly suggests that non-apoptotic signaling triggered by Fas-L in ASCs is caspase-independent. The inventors also found that Fas-L-dependent proliferation of ASCs could be inhibited by ERK and PI3K inhibitors but not by a JNK inhibitor. Importantly, none of the inhibitors significantly abrogated Fas-L-dependent apoptosis in ASCs. Thus, although Fas-L simultaneously induces apoptosis and proliferation of ASCs, the signaling that promotes each functional outcome seems to divert downstream to the Fas receptor.

The present invention demonstrates, for the first time, that Fas-L signaling simultaneously promotes apoptosis and proliferation of freshly isolated and cultured human ASCs, leading to an overall increase in cell expansion and to a phenotypic change. In the context of regenerative medicine, the ability to survive and proliferate under Fas-L-enriched conditions may assist in the survival of SVF cells and ASCs following their clinical administration in vivo.

The present invention thus provides in a first of its aspects a method for propagating mesenchymal stem cells (MSC), the method comprising:
(a) isolating cells from a body tissue or organ; and
(b) incubating the isolated cells obtained in (a) in a growth medium comprising:
   (i) at least one TNF superfamily ligand, and
   (ii) at least one apoptosis inhibitory agent,

thereby obtaining a cell population enriched with MSC.

The term “Mesenchymal stem cells” (MSCs) as known in the art, relates to multipotent stromal cells that can differentiate into a variety of cell types. MSC were recently defined by the International Society of Cellular Therapy as possessing the following characteristics: (1) plastic-adherent when maintained in standard culture conditions; (2) expression of CD105, CD73 and CD90, and lack of expression of CD45, CD34, CD14 or CD11b, CD79a or CD19 and HLA-DR surface molecules (3). MSC differentiate to osteoblasts, adipocytes and chondroblasts in vitro. Methods for isolating, purifying, and expanding human mesenchymal stem cells in culture are known in the art, see for example U.S. Pat. No. 5,486,359 which concerns mainly the isolation of mesenchymal stem cells from bone marrow.

As used herein the term “body tissue or organ” relates to any tissue or organ in the body of a subject that contains mesenchymal stem cells including, but not limited to, adipose tissue, peripheral blood, bone marrow, dental pulp, amniotic fluid, amnion membrane, chorion membrane, chorion villi, decidua, placenta, umbilical cord, cord blood, Wharton's jelly.

In one embodiment, the mesenchymal stem cells of the invention are adipose derived stem cells.

Therefore, in a specific embodiment, the invention provides a method for propagating adipose derived stem cells (ASC), the method comprising:
(a) isolating stromal vascular fraction (SVF) cells from a liposuction aspirate obtained from a subject; and
(b) incubating the isolated cells obtained in (a) in a growth medium comprising:
   (i) at least one TNF superfamily ligand, and
   (ii) at least one apoptosis inhibitory agent,

thereby obtaining a cell population enriched with ASC.

The terms “adipose derived stem cells (ASC)” and “adipose derived mesenchymal stem cells (MSC)” are used interchangeably herein and refer to MSCs that were isolated from adipose tissue.

As used herein, “adipose tissue” refers to a connective tissue composed mostly of adipocytes. In addition to adipocytes, adipose tissue contains the stromal vascular fraction (SVF) of cells including preadipocytes, fibroblasts, vascular endothelial cells, a variety of immune cells such as adipose tissue macrophages, stem cells and endothelial precursor cells.

As used herein the term “Stromal vascular Fraction (SVF)” relates to a preparation of adipose tissue that is a rich source of preadipocytes, mesenchymal stem cells (MSC), endothelial progenitor cells, T cells, B cells, mast cells as well as adipose tissue macrophages.

Methods for isolating adipose-derived stem cells or SVF cells from a liposuction aspirate are known in the art (see for example Bunnell, B. A. et al. (2008) Methods 45(2): 115-120, Schreml et al. (2009) Cytotherapy 11(7): 947-57, Yoshimura et al. (2006) J. of Cellular Physiology 208: 64-76). For example, adipose tissue may be removed from a patient by suction-assisted lipoplasty, ultrasound-assisted lipoplasty, and excisional lipectomy, or a combination thereof. The tissue extraction should be performed in a sterile or aseptic manner.

For suction-assisted lipoplastic procedures, adipose tissue is collected by insertion of a cannula into or near an adipose tissue depot present in the patient followed by aspiration of the adipose tissue into a suction device. A small cannula coupled to a syringe, may be suitable for harvesting relatively moderate amounts of adipose tissue (e.g., from 0.1 ml to a few hundred milliliters). Larger cannulas and automated suction devices may be employed in the procedure if larger volumes of tissue are required.

An exemplary protocol for isolating adipose-derived stem cells or SVF cells from a liposuction aspirate is provided in the Examples section below.

As used here the term “propagating” relates to expansion (expanding), multiplication, proliferation or increase in number in vitro of mesenchymal stem cells isolated from a body tissue or organ, e.g. from liposuction aspirates.

As used herein the term “subject” relates to a mammalian subject, specifically to a human subject.

Optionally, said isolation step (a) comprises exposure to a proteolytic enzyme (e.g. collagenases, e.g. collagenase type I) or a mixture of proteolytic enzymes which breaks down tissue (also termed collagenase digestion). Protocols for performing collagenase digestion of cells are well known in the art, and several compounds for performing collagenase digestion are commercially available (e.g. Celase® GMP by Cytori Therapeutics). Collagenase type I can be added at a range of between 0.1%-0.3%. A neutral protease, e.g. Dispase II can optionally be added to the digestion reaction. For example, as shown in the Examples below, collagenase can be added at a concentration of 0.375 mg/ml for 60 minutes in agitation at 37° C.

As used herein the term a “tumor necrosis factor (TNF) superfamily ligand” refers to a family of more than 40 distinct ligand-receptor systems that are currently recognized as belonging to the TNF superfamily Exemplary ligands that belong to the TNF superfamily and may be used in accordance with the present invention include, but are not limited to Fas-ligand (FasL), TNFα, Trail (Apo2 ligand) and Tweak (Apo3 ligand). Such TNF superfamily ligands may be recombinant polypeptides, biochemically synthesized or purified from cell extracts. They may be administered as monomer, dimer or multimer forms.

Thus in some embodiments the TNF superfamily ligand is selected from the group consisting of FasL, TNFα, TRAIL, TWEAK and a combination thereof.

In a specific embodiment the TNF superfamily ligand is FasL. Non-limiting examples of FasL for use in accordance with the invention include superFasL, APO010 or MegaFasL (R&D), all of which are multimers of FasL. Additional forms of FasL, such as Fc-FasL or FasL monomers, are also encompassed by the invention.

The concentration of the TNF superfamily ligand in the medium is in the range of about 0.1 ng/ml and about 100 ng/ml w/v, for example 0.1 ng/ml, 0.5 ng/ml, 1 ng/ml, 5 ng/ml, 10 ng/ml, 20 ng/ml, 30 ng/ml, 40 ng/ml, 50 ng/ml, 60 ng/ml, 70 ng/ml, 80 ng/ml, 90 ng/ml or 100 ng/ml w/v of the TNF superfamily ligand. In a specific embodiment whereby the TNF superfamily ligand is FasL the medium comprises between about 0.1 ng/ml and about 100 ng/ml w/v FasL, for example 0.1 ng/ml, 0.5 ng/ml, 1 ng/ml, 5 ng/ml, 10 ng/ml, 20 ng/ml, 30 ng/ml, 40 ng/ml, 50 ng/ml, 60 ng/ml, 70 ng/ml, 80 ng/ml, 90 ng/ml or 100 ng/ml w/v FasL. In specific embodiments the medium comprises 25 ng/ml, 50 ng/ml or 100 ng/ml FasL.

In some embodiments, said apoptosis inhibitory agent is selected from the group consisting of a caspase inhibitor, a serine protease inhibitor (e.g. HTRA2), inhibitors of apoptosis proteins (IAP) (e.g. XAF1), NF-kappa B inhibitor, a Smac inhibitor, and a combination thereof.

In one specific embodiment, said caspase inhibitor is Z-VAD-fmk.

In some embodiments, the amount of said apoptosis inhibitory agent is in the range of about 1 μM to about 50 μM, or in the range of about 5 μM to about 25 μM.

As indicated above, the method for propagating mesenchymal stem cells (MSC) or adipose derived stem cells (ASC) comprises incubating the isolated cells in a growth medium comprising a TNF superfamily ligand and an apoptosis inhibitor. As known in the art, the term “incubating” refers to growing and maintaining the cells at an appropriate temperature and gas mixture (typically, 37° C., 5% CO₂ for mammalian cells) in a cell incubator. As used in the context of the present invention, “incubating the isolated cells in a growth medium comprising a TNF superfamily ligand and an apoptosis inhibitor”, means contacting the isolated cells with the TNF superfamily ligand and the apoptosis inhibitor.

In some embodiments the method according to the present disclosure is where the incubation with the TNF superfamily ligand and the apoptosis inhibitor is for at least 1 hour.

In some embodiments, said incubation is for about 1 hour, 2 hours, 3 hours or more, or for about 1, 2, 3, 4, 5, 6, 7, 8, 9, 10, 11, 12, 13, 14, 17, 20, or 23 days.

In one embodiment, said incubation is for 2 hours.

In one embodiment, said incubation is for 4 days.

In one embodiment, said incubation is for 14 days.

In one specific embodiment the TNF superfamily ligand is FasL and the apoptosis inhibitor is Z-VAD-fmk and the incubation is for 4 days.

In some embodiments the medium comprising the TNF superfamily ligand and the apoptosis inhibitor is refreshed every 3 days or every 4 days, namely it is replaced by a fresh medium having the same content.

In some embodiments incubation with the TNF superfamily ligand and the apoptosis inhibitor commences 1-7 days after the cells are isolated. In specific embodiments the method according to the present disclosure is wherein after step (a) the isolated cells are cultured for 1, 2, 3, 4, 5, 6 or 7 days in normal growth medium, followed by incubation with the TNF superfamily ligand and the apoptosis inhibitor.

In one specific embodiment the incubation with the TNF superfamily ligand and the apoptosis inhibitor is performed with mature ASC, namely cells of passage 2 or passage 3 after SVF isolation.

In one embodiment, prior to incubation with FasL, the isolated cells are pre-incubated with a molecule that can affect the cellular response to FasL (i.e. to sensitize the cells to its effect), for example by increasing the expression of Fas. TNFα is an example of such a sensitizing molecule.

Therefore in some embodiments the method according to the present disclosure is wherein prior to incubation with FasL and the apoptosis inhibitor, the isolated cells are pre-incubated with TNFα. In further specific embodiments the pre-incubation with TNFα is performed for 1-4 days.

In certain embodiments, in addition to the TNF superfamily ligand and the apoptosis inhibitor the cells are incubated with an additional active agent selected from the group consisting of a growth factor, a hormone, a cytokine and any combination thereof. In a specific, non-limiting example the cells may be incubated with one or more antagonists of death receptor 3 (DR3, also known as TNFRSF25). Such antagonists include, but are not limited to, anti DR3 antibodies which are commercially available for example from abcam, R&D Systems and other suppliers.

As indicated above, the methods of the invention provide a cell population that is enriched with MSC or ASC. As used herein the term “enriched” refers to enhancing, augmenting or increasing the relative number of MSC or ASC in the cell population to at least about 50%, 55%, 60%, 65%, 70%, 75%, 80%, 85%, 90%, 95% 96%, 97%, 98%, 99% or about 100% of the cell population.

Upon completion of the incubation period with the TNF superfamily ligand and the apoptosis inhibitor the propagated cells are removed from the culture, reseeded and may now differentiate into any one of adipocytes, chondrocytes, osteocytes, fibroblasts, or myocytes depending on the culturing conditions. The conditions for differentiating into each of the desired cell types are well known in the art and include incubation of the cells in a medium that contains specific reagents.

Therefore in some embodiments the method according to the present disclosure is where following incubation with the TNF superfamily ligand and the apoptosis inhibitor, said TNF superfamily ligand and the apoptosis inhibitor are removed and said cells are allowed to differentiate. In other specific embodiments the cells are allowed to differentiate into adipocytes, chondrocytes, or osteocytes, and are termed herein the “differentiated cells”.

As used herein the term “allowed to differentiate” relates to the incubation of the cells under conditions suitable for differentiation into any of the desired cell types. Such conditions are well known in the art and include in particular incubation in a medium that contains a typical set of reagents.

For example, mesenchymal stem cell adipogenesis medium contains reagents that readily differentiate mesenchymal stem cells to an adipogenic lineage (i.e. dexamethasone, IBMX, insulin and indomethacin). Such media are commercially available, for example, Lonza's hMSC Adipogenic Differentiation BulletKit™ Medium or Stemcell Technologies' MesenCult™ Adipogenic Differentiation Medium. Differentiation into adipocytes can then be validated by a variety of methods providing a quantitative value related to the amount of adipogenic differentiation. Other media may be used to induce differentiation to other cell types (e.g. Mesenchymal Stem Cell Chondrogenic Differentiation Medium, Mesenchymal Stem Cell Osteogenic Differentiation Medium or Mesenchymal Stem Cell Neurogenic Differentiation Medium (Promocell). The propagated ASC may also trans-differentiate into cells of germ-origin other than their own, e.g. into cardiomyogenic, endothelial (vascular), pancreatic (endocrine), neurogenic, and hepatic trans-differentiation, while also supporting haematopoesis.

In further embodiments the method according to the present disclosure further comprises transplanting the differentiated cells into a patient in need thereof.

In one embodiment the method according to the invention comprises transplantation of the propagated non-differentiated cells into a patient in need thereof.

As used herein, the term “patient in need thereof” relates to a subject suffering from a disease that may be ameliorated, cured, treated, or having its symptoms alleviated by transplanting the differentiated cells.

Therefore, the differentiated cells may be used for transplantation in a plethora of clinical applications as well as cosmetic applications and plastic surgeries, for example in the following indications that are currently in Phase III/IV clinical trials: Breast reconstruction (restoration), premature ovarian failure, Complex perianal fistula, Anal Fistula and Crohn's disease. ASCs can also be used for cell banking Additional applications include skin regeneration, dermatological conditions, hepatic regeneration, muscle regeneration, blood vessel repair, treatment of orthopedic injuries, bone reconstruction or bone repair, chondrocyte implantation, treatment of meniscus or articular cartilage repair and treatment of osteoporosis.

The differentiated cells of the invention may also be used for immunosuppression. In other embodiments the differentiated cells in accordance with the invention can be used for treating stroke or heart failure.

The transplantation can be allogeneic or autologous.

The term “transplantation” as herein defined refers to transferring the cells to a patient in need thereof by surgery or injection. The donor and the recipient can be a single individual or different individuals. In such cases the transplant is autologous or allogeneic, respectively. When allogeneic transplantation is practiced, regimes for reducing implant rejection should be undertaken. Such regimes are currently practiced in human therapy and are well known in the art.

The following are examples of cells of mesenchymal origin that are used as an external source of cells for replenishing missing or damaged cells of an organ: U.S. Pat. No. 5,736,396 describes introducing culture-expanded lineage-induced mesenchymal stem cells into the original, autologous host, for purposes of mesenchymal tissue regeneration or repair.

U.S. Pat. No. 4,642,120 provides compositions for repairing defects in cartilage and bones. These are provided in gel form either as such, or embedded in natural or artificial bones.

Transplantation of the cells can be performed using any method known in the art and at the physician's discretion based on the patient's therapeutic indication. For example, the differentiated cells may be injected at the site of a tissue defect (e.g. a skeletal defect), or incubated in vitro with a biocompatible scaffold or matrix and implanted with the scaffold at the site of the defect.

The cells of the invention may also be employed in gene therapy. For successful long-term gene therapy, a high frequency of genetically modified cells stably expressing a desired foreign gene is required. Accordingly, the differentiated cells of the present invention can be modified to express a gene product.

As used herein, the term “gene product” refers to proteins, peptides and functional RNA molecules (i.e. polynucleotides). Examples of such gene products include insulin, amylase, protease, lipase, trypsinogen, chymotrypsinogen, carboxypeptidase, ribonuclease, deoxyribonuclease, triaclyglycerol lipase, elastase, amylase, blood clotting factors such as blood clotting Factor VIII and Factor IX, UDP glucuronyl transferase, ornithine transcarbanoylase, and cytochrome p450 enzymes, and adenosine deaminase, for the processing of serum adenosine or the endocytosis of low density lipoproteins, serum thymic factor, thymic humoral factor, thymopoietin, gastrin, secretin, cholecystokinin, somatostatin, serotonin, and substance P.

In another one of its aspects the present disclosure provides an adipose stem cell (ASC) growth medium or a mesenchymal stem cell (MSC) growth medium comprising a cell culture medium, at least one TNF superfamily ligand and at least one apoptosis inhibitory agent.

A “cell culture medium” or a “growth medium” as herein defined refers to a liquid or gel designed to support the growth of cells. Different types of media are used for growing different types of cells. Examples for cell culture media encompass Dulbecco's Modified Eagle Medium (DMEM) and variants thereof, F10 Nutrient Mixture, Ham's F12. Nutrient Mixture, Minimum Essential Media (MEM), RPMI Media 1640 and Iscove's Modified Dulbecco's Medium (IMDM).

All these media are well known and commercially available. For example, DMEM typically includes amino acids, inorganic salts, glucose, phenol red, HEPES, sodium pyruvate, and vitamins. The media are often supplemented with antibiotic/antimycotic agents such as penicillin, streptomycin and amphotericin B. The media may include varying glucose contents. In one embodiment the medium has high glucose content, e.g. High glucose DMEM by Gibco.

The cell culture medium may further comprise serum or serum substitutes.

The term “serum” or “sera” in the context of the present disclosure refers to a supplement added to cell culture media that provides a broad spectrum of macromolecules, carrier proteins for lipoid substances and trace elements, attachment and spreading factors, low molecular weight nutrients, and hormones and growth factors. Typically sera are isolated from mammalian blood preparations. A non-limiting example of a serum suitable for use in connection with the present invention is Fetal Calf serum (FCS) or Fetal Bovine Serum (FBS). Serum substitutes may also be used. The term “serum substitute” relates to serum replacements containing proteins necessary to support cell growth in culture, for example serum substitutes may comprise purified, preferably heat-treated serum albumin, transferrin and insulin. Such proteins may be of any origin, they may be for example mammalian proteins, e.g. bovine serum albumin, transferrin and insulin. Such serum substitutes are commercially available, for example from Sigma-Aldrich. The concentration of the serum in the medium is in the range commonly used for growing cells, namely between about 1% and about 20%. In a specific embodiment the concentration of the serum is about 10% volume/volume. In a further specific embodiment the medium comprises 10% FCS or FBS, e.g. Fetal Bovine Serum by Hyclone™.

The TNF superfamily ligands used in the context of the MSC growth medium or the ASC growth medium are as herein defined, e.g. FasL, TNFα, TRAIL (Apo2 ligand) or TWEAK (Apo3 ligand). The concentration of the TNF superfamily ligand in the medium is in the range of about 0.1 ng/ml and about 100 ng/ml w/v, for example 0.1 ng/ml, 0.5 ng/ml 1 ng/ml, 5 ng/ml, 10 ng/ml, 20 ng/ml, 30 ng/ml, 40 ng/ml, 50 ng/ml, 60 ng/ml, 70 ng/ml, 80 ng/ml, 90 ng/ml or 100 ng/ml w/v of the TNF superfamily ligand. In a specific embodiment the TNF superfamily ligand is FasL. In a further specific embodiment the medium comprises between about 0.1 ng/ml and about 100 ng/ml w/v FasL, for example 0.1 ng/ml, 0.5 ng/ml, 1 ng/ml, 5 ng/ml, 10 ng/ml, 20 ng/ml, 30 ng/ml, 40 ng/ml, 50 ng/ml, 60 ng/ml, 70 ng/ml, 80 ng/ml, 90 ng/ml or 100 ng/ml w/v FasL.

The apoptosis inhibitory agents used in the context of the MSC growth medium or the ASC growth medium are selected from the group consisting of a caspase inhibitor, a serine protease inhibitor (e.g. HTRA2), inhibitors of apoptosis proteins (IAP) (e.g. XAF1), NF-kappa B inhibitor, a Smac inhibitor or a combination thereof.

In one specific embodiment, said caspase inhibitor is Z-VAD-fmk.

In some embodiments, the concentration of said apoptosis inhibitory agent in the medium is in the range of about 1 μM to about 50 μM, or in the range of about 5 μM to about 25 μM.

The MSC or ASC growth medium of the invention may further comprise additional components suitable for cell growth including but not limited to growth factors, cytokines, antibiotics and carriers.

The MSC or ASC growth medium of the invention may be prepared and stored at 4° C. until use and warmed to 37° C. prior to use.

In another aspect, the present invention provides an article of manufacture comprising:

a. a vessel containing an adipose stem cell growth medium or mesenchymal stem cell growth medium, wherein said growth medium comprises a cell culture medium, a TNF superfamily ligand and an apoptosis inhibitory agent; and

b. instructions for using the growth medium for expanding adipose stem cells or mesenchymal stem cells in vitro.

In one embodiment, said growth medium further comprises serum or serum substitutes.

The term “vessel” as herein defined refers to a container, bag, beaker, bottle, bowl, box, bioreactor, can, flask, or vial that may be used for containing and storing the mesenchymal stem cell growth medium as herein defined.

The term “about” as herein defined indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range.

### EXAMPLES

Without further elaboration, it is believed that one skilled in the art can, using the preceding description, utilize the present disclosure to its fullest extent. The following preferred specific embodiments are, therefore, to be construed as merely illustrative, and not limitative of the claimed invention in any way.

Standard molecular biology protocols known in the art not specifically described herein are generally followed essentially as in Sambrook & Russell, 2001.

Standard medicinal chemistry methods known in the art not specifically described herein are generally followed essentially in the series “Comprehensive Medicinal Chemistry” by various authors and editors, published by Pergamon Press.

### Example 1: Simultaneous SVF Cell Proliferation and Apoptosis Following Fas-L Treatment

In order to evaluate the effect of Fas-L treatment on stromal vascular fraction (SVF) cells, SVF cells were obtained from liposuction aspirates, as described below.

The human SVF cells were cultured for 14 days in normal growth medium as specified below in the presence versus absence of increasing Fas-L concentrations, and the cell yield and apoptosis rate were measured. The experiment was repeated 3 times using cells from 3 independent patients. As shown in FIGS. 1A and 1B, no response to Fas-L concentrations of up to 25 ng/ml was recorded. In contrast, at concentrations of 25 ng/ml and above, SVF cells demonstrated a dose-dependent increase in apoptosis rates (FIG. 1B). Nonetheless, the cell counts were double or more (FIG. 1A). FIG. 1C and FIG. 1D depict apoptosis induced in control SVF cells (without Fas-L treatment) (1C) and in cells treated with 50 ng/ml Fas-L (1D) as calculated by propidium iodide (PI) cell-cycle flow cytometry analysis. The number of Apoptotic cells increased from 5.4% to 20.5% following incubation with 50 ng/ml FasL. FIGS. 1E and 1F show images of cultured SVF cells either with 50 ng/ml Fas-L (1F) or without Fas-L (control) (1E). FIG. 1G is a graphical representation of annexin/PI flow cytometry analysis of floating cells of treated cells (marked by arrow in FIG. 1F). FIGS. 1H and 1I is a graphic representation of CD95 (Fas) expression on Fas-L treated and untreated cells as examined by Flow cytometry analysis. A doubled cell yield following 50 ng/ml Fas-L treatment was demonstrated in SVF cells extracted from 10 independent patients (Table 1). Thus, Fas-L treatment (≥25 ng/ml) promotes both apoptosis and proliferation of SVF cells in culture.

TABLE 1
Increased cell yield following Fas-L treatment of SVF cells

| | Untreated ASC | Fas-L treated ASC | Ratio Fas-L/ |
|---|---|---|---|
| Patient No. | Cell no. × 10⁶ | Cell no. × 10⁶ | control |
| 1 | 0.22 | 0.41 | 1.9 |
| 2 | 0.16 | 0.23 | 1.4 |
| 3 | 0.09 | 0.24 | 2.7 |
| 4 | 0.137 | 0.23 | 1.7 |
| 5 | 0.059 | 0.13 | 2.2 |
| 6 | 0.12 | 0.19 | 1.6 |
| 7 | 0.126 | 0.209 | 1.7 |
| 8 | 0.149 | 0.321 | 2.2 |
| 9 | 0.069 | 0.109 | 1.6 |

### Example 2: Fas-L Treatment of SVF Cells Leads to an Increase in the CD105-Low and CD73-High Cell Populations of Passage 0 and 1 ASCs

Next, the effect of Fas-L treatment on the cells' phenotype was examined. To this end, the surface marker profile of Fas-L-treated (50 ng/ml) SVF cells at passage 0 and 1 was compared to that of untreated controls, using a 7-color flow cytometry panel. The experiment was repeated 2 times using cells from 2 independent patients giving the same trend.

As expected, at passage 0, fewer than 2% of the untreated cells expressed CD45, CD31 and CD34, close to a 100% expressed MSC cell markers CD29 and CD73 and CD105 (FIGS. 2A-H). A similar expression profile was recorded at passage 1 in untreated cells (FIGS. 2I-L). Despite a significant similarity in the surface marker expression pattern of untreated and Fas-L treated passage 0 cells, a higher percentage of CD105-positive cells expressing low levels of CD105 was noted in Fas-L treated cells as compared to untreated control cells (CD105-low (66.9% compared to 50.1% respectively). In addition Fas-L-treated passage 0 cells displayed a higher percentage of CD73-positive cells expressing high CD73 levels (CD73 high) (88.3% compared to 50.4%), as compared to untreated control cells (FIGS. 2A-H). A further increase in the relative percentages of these two subpopulations was noted in passage 1, with 89.6% of Fas-L-treated CD105 positive cells demonstrating CD105-low and 92.8% CD73 positive cells demonstrating CD73-high, as compared to only 49.9% and 52.5%, respectively, of the passage 1 untreated control (FIGS. 2I-L). Thus, Fas-L treatment of SVF cells leads to the enrichment of CD105-low and CD73-high cells.

### Example 3: Fas-L-Treated Cultured SVF Cells Display Similar Fat Differentiation and Higher Bone Differentiation Compared to Untreated Cells

Different ASC subpopulations, characterized by distinct surface marker expression, are known to demonstrate different differentiation potentials. The differentiation of Fas-L-treated versus untreated passage 0 ASCs to fat and bone was compared. Human SVF cells were cultured for 14 days under normal culture conditions or with 50 ng/ml Fas-L. Treated and untreated cells were than passaged to P1 and induced to undergo fat or bone differentiation using designated differentiation media at P1. Differentiation into bone and fat was detected by Alizarin red and Oil red O staining, respectively. Cells which differentiated to fat (FIGS. 3A-C) and bone (FIGS. 3D-F) were then photographed and the stain was extracted and quantified. The experiment was repeated 3 times using cells from 3 independent patients giving the same trend.

The comparison revealed similar fat differentiation in Fas-L-treated and untreated cells, but enhanced bone differentiation following Fas-L treatment.

### Example 4: Fas-L Treatment of SVF Cells Promotes the Production of Large CFU-Fs

Next, the capacity of Fas-L-treated and untreated SVF cells to form colony forming unit fibroblasts (CFU-Fs) was evaluated. Human SVF cells were cultured at low densities, for 21 days, under normal culture conditions or with 50 ng/ml Fas-L. Colonies were then stained by Giemsa and counted. Colonies were photographed and the total number of colonies and of large colonies (>100 cells) was compared between Fas-L-treated and untreated cells. The experiment was repeated 2 times using cells from 2 independent patients giving the same trend.

While at a first glance, it seemed that Fas-L-treated cells formed significantly more CFU-Fs than untreated cells (FIG. 4A), careful microscopic evaluation revealed that the CFU-Fs population was comprised of both large and small CFU-Fs, where the large colonies were of more than a 100 cells (FIGS. 4B and 4C bottom). As can be seen in FIGS. 4D and 4E while the total number of colonies did not reveal a significant difference between Fas-L-treated and untreated cells, a significantly higher number of large colonies was found in Fas-L-treated SVF cells compared to untreated cells.

### Example 5: Fas-L Treatment of Passage 3 ASCs Results in a Phenotype Shift Similar to that Observed in SVF Cells

All the experiments thus far evaluated the effect of Fas-L treatment on early SVF cells, immediately following their isolation from fat. In order to examine whether Fas-L can induce a similar effect when added to “mature” ASCs, passage 3 (P3) cells were cultured for 6 days under normal conditions or in the presence of increasing concentrations of Fas-L. Cell numbers (FIG. 5A) and sub-G1 proportions (FIGS. 5B-5D) were examined by standard cell counts and by propidium iodide (PI) cell-cycle flow cytometry analysis, respectively. The experiment was repeated 3 times using cells from 3 independent patients. Data are presented as the mean±standard deviation. In addition, surface marker expression of CD29, CD105 and CD73 was examined by flow cytometry in the P3 ASCs cultured under normal culture conditions or with 50 ng/ml Fas-L.

As with SVF cells, Fas-L treatment led to a concentration-dependent increase in proliferation (FIG. 5A) and to an increase in the CD105-low and CD73-high populations (FIGS. 5E-5J) of P3 ASCs. Fas-L also induced apoptosis of P3 ASCs (FIGS. 5B-5D), but at a significantly lower rate compared to that seen following Fas-L treatment of SVF cells.

### Example 6: Caspase Inhibition Attenuates Fas-L-Dependent Apoptosis but not Proliferation

The dual effect of Fas-L on the apoptosis and proliferation of ASCs, can be mediated solely by canonical Fas-L signaling or through two distinct pathways. To distinguish between these possibilities, ASCs were either left untreated, treated with Fas-L only or treated simultaneously with Fas-L and with the pan-caspase inhibitor Z-VAD-fink, for 4 days. Importantly, to better evaluate the apoptotic cue of Fas-L, a different Fas-L derivative (mega-Fas-L), which induces high rates of apoptosis in passage 3 ASCs, was used in the current experiments. Sub-G1 proportions and cell numbers were determined by PI cell-cycle flow cytometry analysis and standard cell counts respectively. The experiment was repeated 3 times using cells from 3 independent patients.

As expected, Fas-L treatment led to a sharp increase in ASC apoptosis, as demonstrated by an increase in the sub-G1 population of treated cells (FIGS. 6A and C-E). While apoptosis was considerably attenuated by the addition of Z-VAD-fmk (FIGS. 6A and C-E), Fas-L-induced ASC proliferation was not affected, and the cell yield doubled over the 4-day treatment period (FIG. 6B). No such increased expansion was observed in cells treated with Z-VAD-fmk only. In the current experiments treatment of Fas-L only did not demonstrate an increased cell yield compared to untreated cells (FIG. 6B). This was due to the shorter duration of this experiment (4 days) as compared to previous experiments (7-14 days) and not to the change in the Fas-L derivative as treatment with mega-Fas-L for 7 days led to a doubled cell yield (Table 2) (average 1.9, SD 0.2).

TABLE 2
Increased cell yield following Mega-
Fas-L treatment of Passage 1-3 ASCs

| | | | Untreated | Fas-L treated | |
|---|---|---|---|---|---|
| | | | ASC | ASC | Ratio |
| Patient | Mega-Fas-L | | Cell | Cell | Fas-L/ |
| no. | ng/ml | Passage | no. × 10⁶ | no. × 10⁶ | control |
| 1 | 20 | P3 | 0.135 | 0.249 | 1.8 |
| 2 | 30 | P1 | 0.71 | 1.56 | 2.2 |
| 3 | 15 | P1 | 0.63 | 1.19 | 1.9 |
| 4 | 30 | P3 | 0.66 | 1.2 | 1.8 |

### Example 7: Inhibition of PI3K and MAP Kinases ERK-1/2 but not of JNK Suppresses Fas-L-Induced Proliferation of ASCs

To identify the intracellular pathways that control Fas-L-induced proliferation in ASCs, various signaling pathways which were previously shown to be activated by Fas-L [16-20], were independently inhibited. These include PI3K/Akt, ERK-1/2 and JNK signaling pathways.

Accordingly, cultured Passage 2-4 ASCs were treated with a combination of mega Fas-L, Z-VAD-fmk, LY294002 (PI3K inhibitor), AZD6244 (ERK-1/2 inhibitor) and SP600125 (JNK inhibitor) for 4 days.

Inhibitor concentrations were calibrated to the maximal doses which did not interfere with basal ASC proliferation (FIGS. 7A-C). After 4 days, cells were removed from plates by trypsin and cells were counted using a standard cell counter. The experiment was repeated 3 times using cells from 3 independent patients. After 4 days in culture with both Fas-L and either PI3K (FIG. 7A) or ERK-1/2 (FIG. 7B) inhibitors, reduced ASC counts were measured as compared to cells treated with Fas-L only (FIGS. 7A and B respectively), indicating inhibition of the pro-proliferative but not of the pro-apoptotic effect of Fas-L by the inhibitors. These findings were confirmed upon culture of ASCs with Fas-L, Z-VAD-fmk and either PI3K (FIG. 8A) or ERK-1/2 (FIG. 7B) inhibitors, which yielded cell quantities comparable to untreated control, while a ˜doubled cell yield was demonstrated following treatment with Fas-L and Z-VAD-fmk (FIGS. 8A and B). In contrast, JNK inhibition concomitant to Fas-L stimulation, yielded cell numbers similar to those obtained following Fas-L treatment only (FIG. 7C). Similarly, the simultaneous addition of Fas-L, Z-VAD-fmk and a JNK inhibitor resulted in population doubling as obtained upon treatment with Fas-L and Z-VAD (FIG. 8C).

### Materials and Methods

Materials

Recombinant human super Fas-L was obtained from Enzo Life Sciences (L\ddot{ }orrach, Germany) and recombinant human mega Fas-L from AdipoGen (San Diego, Calif., USA). zVAD.fmk (pan-caspase inhibitor), LY294002 (PI3K inhibitor) and SP600125 (JNK inhibitor) were obtained from Merck Biosciences (Darmstadt, Germany) and AZD6244 (ERK-1/2 inhibitor) was obtained from Cayman Chemical (Michigan, USA).

The following antibodies were used for flow cytometry stainings: anti-human CD95 (APO-1/Fas) APC and APC mouse IgG1 k isotype control (eBioscience San Diego, Calif.), anti-human CD31 APC and mouse IgG1 isotype control APC, anti-human CD34 PE and mouse IgG1 k Isotype control PE (PeproThec London, UK), anti-human CD29 Alexa Fluor 488 and Alexa Fluor 488 mouse IgG1 k isotype control, anti-human CD105 PerCP/Cy5.5 and PerCP/Cy5.5 mouse IgG1 k Isotype control, anti-human 73 PE/Cy7 and PE/Cy7 mouse IgG1 k isotype control (BioLegend (San Diego, Calif.), anti-human CD45 BD Horizon BV650 and BV650 mouse IgG1, k isotype control BD Biosciences (San Jose, Calif.).

Human Primary SVF Cell Isolation

Subcutaneous adipose tissue samples were obtained from patients undergoing plastic surgery. All procedures were performed in accordance with the Declaration of Helsinki and approved by the ethics committee of Tel Aviv Sourasky Medical Center. Written, informed consent was obtained from all patients in advance. All samples were waste materials collected as a byproduct of surgery. The mean age of the patients was 46.1+-11.7 years, the mean BMI was 29.3+-4.8 kg/m².

SVF cells were isolated from the subcutaneous human lipoaspirates using 0.1% collagenase (Sigma, St. Louis, Mo., USA), and separated from the fat by centrifugation (15 min, 400 g).

Cell Culture

Human primary SVF cells from adipose tissue were maintained in their undifferentiated state in high-glucose Dulbecco's modified Eagle's medium (DMEM) (Gibco, Paisley, Scotland, UK), supplemented with 10% fetal calf serum (FCS) (Thermo Scientific HyClone, Tauranaga, New Zealand), 60 μg/ml penicillin, 100 μg/ml streptomycin, 50 μg/ml kanamycin, 1 mM sodium pyruvate, 2 mM L-glutamine and non-essential amino acids, under 10% CO₂ and atmospheric oxygen conditions. Medium was changed twice a week, and cells were passaged once they reached confluence.

Cell Proliferation

SVF were seeded into 6-well plates (150,000 cells/well) and cultured for 14 days in either normal growth medium or in growth medium supplemented with different Fas-L concentrations. Medium was refreshed four times during this 14 days culture.

Mature ASCs (P2-P3 ASCs) were seeded into 6 cm plates (90,000 cells/plate) and allowed to attach and grow for 24 h. Then, medium was changed to fresh normal growth medium or growth medium supplemented with different Fas-L concentrations, and cells were further cultured for 7 days. Medium was refreshed twice during the experiment period.

To test the effects of inhibitors on proliferation of mature ASCs (P2 and P3 ASCs), cells were seeded into 24-well plates (10.000 cells/well) and allowed to attach and grow for 24 h. Then, medium was exchanged for fresh normal growth medium or growth medium supplemented with Fas-L or with Fas-L and one of the inhibitors. Cells were then cultured for 4 days.

For all treatments, duplicate wells/plates were prepared.

At the end of the incubation period, adherent cells were trypsinized, collected and counted with a TC10 Automated Cell Counter (Bio-rad).

Flow Cytometry

Surface Marker Analysis

For surface marker analysis, cells were harvested and incubated (1 hour, in the dark) with a 7-color panel containing anti-CD31, anti-CD34, anti-CD29, anti-CD105, anti-human-73 and anti-CD45 antibodies. To exclude dead cells, the samples were stained with ViViD (violet viability dye, Molecular Probes, Invitrogen, Eugene, Oreg., USA), according to the manufacturer's protocol. CD95 staining was performed using a 2-color panel containing anti-CD95 antibody and ViViD. All antibodies were used at the dilution recommended by the manufacturer. Appropriate single-stained and isotype controls samples were prepared and analyzed.

Cell Cycle Analysis

Cells were harvested and fixed with 70% ethanol/PBS, treated with RNaseA 0.4 mg/ml (Sigma) and stained with propidium iodide (PI) 0.1 mg/ml (Sigma).

Annexin/PI Analysis

Cells were stained with an Annexin-APC/PI using a dedicated kit (BioLegend).

All labeled cells were analyzed using a FACS Canto II flow cytometer (Becton Dickinson, San Jose, Calif., USA). At least 10.000 events were recorded. Data analysis was performed using the FlowJo software (Tree Star, Ashland, Oreg., USA).

Differentiation

Adipogenic Differentiation

Confluent cells were cultured in adipogenic medium containing 10 μg/ml insulin, 1×10-6 M dexamethasone, 0.5 mM 3-isobutyl-1-methylxanthine (IBMX) and 50 μM indomethacin (all from Sigma). The induction medium was replaced every 3-4 days. After 21 days, the cells were fixed with 4% formalin (20 min at room temperature (RT)) and stained with 0.5% Oil Red (Sigma) (10 min, at RT). Following the staining, cells were photographed with an Olympus IX71 microscope (Olympus, Tokyo, Japan), equipped with a DP73 camera. Oil Red O was then extracted with 4% IGEPAL (Sigma) in isopropanol, and then quantified at 520 nm using a TECAN Infinite M200 plate-reader (TECAN, Männedorf, Switzerland).

Osteogenic Differentiation

Confluent cells were cultured in StemPro osteogenesis differentiation medium (Gibco). The induction medium was replaced every 3-4 days. After 21 days, the cells were fixed with 4% formalin (20 min at RT) and stained with 2% Alizarin red (Sigma), pH 4.2 (10 min, at RT). Photographs were taken using an Olympus IX71 microscope with a DP73 camera. Alizarin red was extracted with extraction solution (0.5N HCL/5% SDS) and quantified at 415 nm using a TECAN Infinite M200 plate reader (TECAN, Männedorf, Switzerland).

CFU-F Assay

SVF cells (2,000) were seeded on a 6-cm dish and cultured for 21 days under normal culture conditions or with medium supplemented with 50 ng/ml Fas-L. Colonies were then fixed with methanol, stained with Giemsa (Sigma) and counted to evaluate CFU-F formation. The total number of colonies and of large colonies (>100 cells) was quantified (using an Olympus IX71 microscope) and compared between Fas-L-treated and untreated cells. These experiments were performed in triplicates.

Statistical Evaluation

The statistical analysis was performed using the Minitab 18 software (Minitab Inc., Pennsylvania). The data were subjected to one-way analysis of variance (ANOVA) followed by Dunnett's (for comparison to control group) or Tukey's multicomparison test. Univariate data were analyzed using paired t test. p≤0.05 was considered statistically significant.

1-40. (canceled)

41. A method for propagating mesenchymal stem cells (MSC), the method comprising:
(a) isolating cells from a body tissue or organ; and
(b) incubating the isolated cells obtained in (a) in a growth medium comprising:
(i) at least one TNF superfamily ligand, and
(ii) at least one apoptosis inhibitory agent,
thereby obtaining a cell population enriched with MSC.

42. A method for propagating adipose derived stem cells (ASC), the method comprising:
(a) isolating stromal vascular fraction (SVF) cells from a liposuction aspirate; and
(b) incubating the isolated cells obtained in (a) in a growth medium comprising:
(i) at least one TNF superfamily ligand, and
(ii) at least one apoptosis inhibitory agent,
thereby obtaining a cell population enriched with ASC.

43. The method of claim 41 wherein said MSC are adipose derived stem cells and wherein said step (a) of isolating cells from a body tissue or organ comprises isolating stromal vascular fraction (SVF) cells from a liposuction aspirate.

44. The method of claim 42 wherein prior to step (b) said SVF cells are maintained in culture for at least two passages.

45. The method of claim 41, wherein said TNF superfamily ligand is selected from the group consisting of Fas-ligand (FasL), tumor necrosis factor (TNF) α, TNF-related apoptosis-inducing ligand (TRAIL), tumor necrosis factor-like weak inducer of apoptosis (TWEAK), and a combination thereof.

46. The method of claim 41 wherein said growth medium further comprises an additional active agent selected from the group consisting of a growth factor, a hormone, a cytokine and any combination thereof.

47. The method of claim 41, wherein said incubation is for about 1 hour, 2 hours, 3 hours or more, or for about 1, 2, 3, 4, 5, 6, 7, 8, 9, 10, 11, 12, 13, 14, 17, 20, or 23 days.

48. The method of claim 47 wherein said incubation is for 14 days and wherein the medium is refreshed every 3 days or every 4 days.

49. The method of claim 41 wherein said apoptosis inhibitory agent is selected from the group consisting of a caspase inhibitor (e.g. Z-VAD-fmk), a serine protease (e.g. HTRA2) inhibitor, inhibitors of apoptosis proteins (IAP) (e.g. XAF1), NF-kappa B inhibitor, a Smac inhibitor, and a combination thereof.

50. The method of claim 41, wherein the amount of said apoptosis inhibitory agent is in the range of about 1 μM to about 50 μM, or in the range of about 5 μM to about 25 μM.

51. The method of claim 41, wherein the concentration of said at least one TNF superfamily ligand is in the range of about 0.1 ng/ml to about 100 ng/ml.

52. The method of claim 41 wherein following incubation with said at least one TNT superfamily ligand and the apoptosis inhibitory agent, said at least one TNF superfamily ligand and the apoptosis inhibitory agent are removed and said cells are allowed to differentiate.

53. The method of claim 52 wherein said cells are allowed to differentiate into adipocytes, chondrocytes, or osteocytes.

54. The method of claim 41 further comprising transplanting said differentiated or undifferentiated cells into a patient in need thereof.

55. The method of claim 54 wherein said cells differentiate into fat tissue and wherein said patient is in need of breast restoration, or wherein said cells differentiate into bone tissue and said patient is in need of treatment of orthopedic injuries, bone reconstruction or bone repair, or wherein said cells differentiate into cartilage tissue and said patient is in need of chondrocyte implantation, treatment of meniscus, rotator cuff or an articular cartilage repair, or wherein said patient is in need of a cosmetic procedure.

56. The method of claim 54 wherein said transplanting said differentiated cells is for treating a dermatological condition, or for alleviating or treating an immune-related disease or for organ or tissue transplantation, or for treating cardiac disorders, or for treating anal or perianal fistulas in a Crohn's disease patient, or for blood vessel repair.

57. The method of claim 41 wherein said cells are allogeneic or autologous.

58. An adipose stem cell (ASC) or mesenchymal stem cell (MSC) growth medium comprising a cell culture medium, a TNF superfamily ligand and an apoptosis inhibitory agent.

59. An article of manufacture comprising:
a. a vessel containing an adipose stem cell or mesenchymal stem cell growth medium, wherein said growth medium comprises a cell culture medium, a TNF superfamily ligand and an apoptosis inhibitory agent; and
b. instructions for using the growth medium for expanding adipose stem cells or mesenchymal stem cells in vitro.

60. An enriched population of cells obtained by the method of claim 41.
